(19) United States
(12) Patent Application Publication
(10) Pub. No.: US 2003/0018308 A1
(43) Pub. Date: Jan. 23, 2003

(54) Drug-adding tube of infusion set

(76) Inventor: Ming-Hsiung Tsai, Taichung (TW)

Correspondence Address: Ming-Hsiung Tsai, P.O. BOX 2103, Taichung, TW

(21) Appl. No.: 10/162,476

(22) Filed: Jun. 5, 2002

(30) Foreign Application Priority Data
Jul. 17, 2001 (TW) 90212015

(51) Int. Cl. A61M 5/00

(52) U.S. Cl. 604/284; 604/257

(57) ABSTRACT

A drug-adding tube of infusion set, including a tubular main body two ends of which are respectively connected with the infusion set. At least one drug-adding tube is obliquely disposed on the main body. The drug-adding tube is formed with a neck section adjacent to the main body. The neck section has reduced inner diameter. A rubber-made valve plug is plugged in an opening of the drug-adding tube. The valve plug is formed with an insertion port. A valve body is formed in the valve plug to interrupt the insertion port. The center of the valve body is formed with a split passing through the valve body. In normal state, due to the valve body's own resilience, the split is compressed and sealed. When a syringe frees from any needle is inserted into the insertion port, the syringe stretches open the split of the valve body to free the same.

### BACKGROUND OF THE INVENTION

The present invention is related to an improved drug-adding tube of infusion set. The drug-adding tube is formed with a neck section having tapered inner diameter. A valve plug formed with an insertion port is plugged in an opening of the drug-adding tube. In normal state, the valve plug is sealed. When a syringe free from any needle is inserted into the insertion port, the syringe stretches and frees the valve body to conveniently inject medicine into the drug-adding tube and ensure the occupational safety of all medical professionals.

A conventional infusion set opens with two ends. One end of the infusion set connects with a plastic needle can thrust into a plastic infusion bag, the other end connects to an intravenous catheter. The intravenous catheter penetrates into human body. A reserving-bag is disposed on a section of the infusion set near the plastic needle. A drug-adding tube is disposed on another section of the infusion set near the intravenous catheter for adding medicine into the infusion set with a syringe.

FIG. 8 shows a conventional drug-adding tube structure of an infusion set. One end of the drug-adding tube 81 has a drug-adding port 811. The other end of the drug-adding tube 81 opposites to the drug-adding port 811 is an outlet 812 connected with the cannular 83. On the other side of the drug-adding tube 81 has an oblique bypass 82 connected with the cannular 83. The dropping medicine flows from the bypass 82 through the drug-adding tube 81 and then flows out to the outlet 812. A rubber membrane 85 is fastened on the drug-adding port 811 by a cap 84 to seal the port 811.

Traditionally when adding medicine into the infusion set is needed, a medical worker thrusts the needle 91 of a syringe 9 through the rubber membrane 85 to inject the medicine from the syringe 9 into the drug-adding tube 81 and then infuses into human body. After the needle 91 is extracted, the rubber membrane 85 by means of its own resilience extends to compress and seal the thrust hole. However, the above operation has some dangers as follows:

1. A worker must hold the syringe 9 with one hand and hold the drug-adding tube 81 with the other hand to thrust the needle 91 through the rubber membrane 85. The worker may incautiously impale himself/herself with the needle 91 and get infected.

2. When the needle 91 thrusts through the rubber membrane 85, the rubber chips are produced. The rubber chips may enter the vessels and cause harms to human body.

3. A plastic infusion bag may use for several hours. During this period, many additional medicines may be injected into the drug-adding tube 81. Therefore, the needle 91 will repeatedly thrust through the rubber membrane 85. As a result, many holes will be formed on the rubber membrane 85 and the holes may be enlarged and can be hardly truly sealed. As a result, the medicine may leak through the holes or even the external bacteria may enter the infusion set through the holes to cause infection.

4. When thrusting the needle 91 through the rubber membrane 85, the worker must be very careful so as to avoid impalement of the hand holding the drug-adding tube 81. Therefore, a worker can hardly quickly add medicines into the infusion set as necessary ( such as in an emergency).

In order to solve the above problems, the needle 91 of the syringe 9 should be removed from the syringe 9. The rubber membrane 85 can be previously formed with a fissure for the syringe 9 free from the needle 91 to insert into the drug-adding tube 81. However, The syringe 9 free from the needle 91 has an opening with a considerably enlarged diameter. With such syringe 9, in case of incubation, the infusion speed will be too fast. Therefore, a medical worker needs to be retrained of a new injection mode. Moreover, the fissure of the rubber membrane 85 will be much larger than the thrust hole thereof. Accordingly, problems of leakage and infection will inevitably take place.

### SUMMARY OF THE INVENTION

It is therefore a primary object of the present invention to provide an improved drug-adding tube of infusion set. The drug-adding tube is obliquely disposed on a main body. The drug-adding tube is formed with a neck section adjacent to the main body. The neck section has tapered inner diameter. A valve plug is plugged in an opening of the drug-adding tube. The valve plug is formed with an insertion port. A valve body is formed in the valve plug to interrupt the insertion port. The center of the valve body is formed with a split passing through the valve body. In normal state, due to the valve body's own resilience, the split is compressed and sealed. When a syringe frees from any needle is inserted into the insertion port, the syringe stretches and splits the valve body. Thereafter, the medicine can be conveniently injected into the drug-adding tube and the safety of a medical worker can be ensured.

It is a further object of the present invention to provide the above drug-adding tube of infusion set in which a connecting string integrally connects a cap with the drug-adding tube. The cap has a boss section which can be inserted into the insertion port of the valve plug to protect and truly seal the drug-adding tube to avoid contamination and infection.

The present invention can be best understood through the following description and accompanying drawings wherein:

### BRIEF DESCRIPTION OF THE DRAWINGS

FIG. 1 is a perspective assembled view of the present invention;

FIG. 2 is a perspective exploded view of the present invention;

FIG. 3 is a sectional view taken along line 3-3 of FIG. 1;

FIG. 4 shows that the drug-adding tube of the present invention is disposed on an infusion set;

FIG. 5 is a sectional view showing the use of the present invention;

FIG. 6 is a sectional view of a second embodiment of the present invention;

FIG. 7 shows a third embodiment of the drug-adding tube of the present invention which is disposed on a reserving bag; and

FIG. 8 shows a conventional drug-adding tube of an infusion set.

### DETAILED DESCRIPTION OF THE PREFERRED EMBODIMENTS

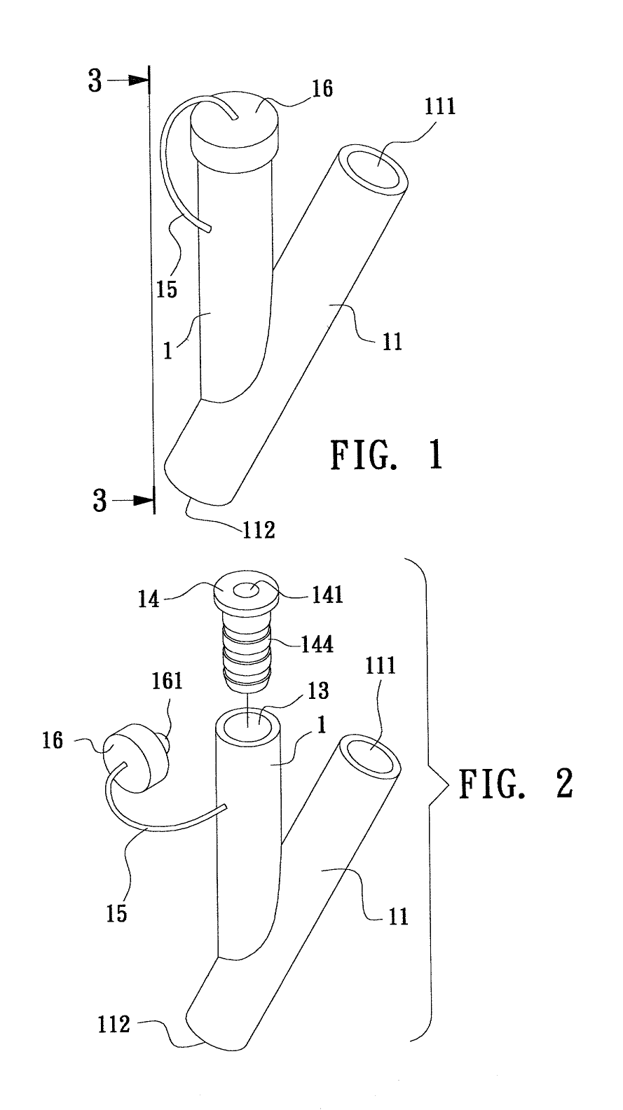

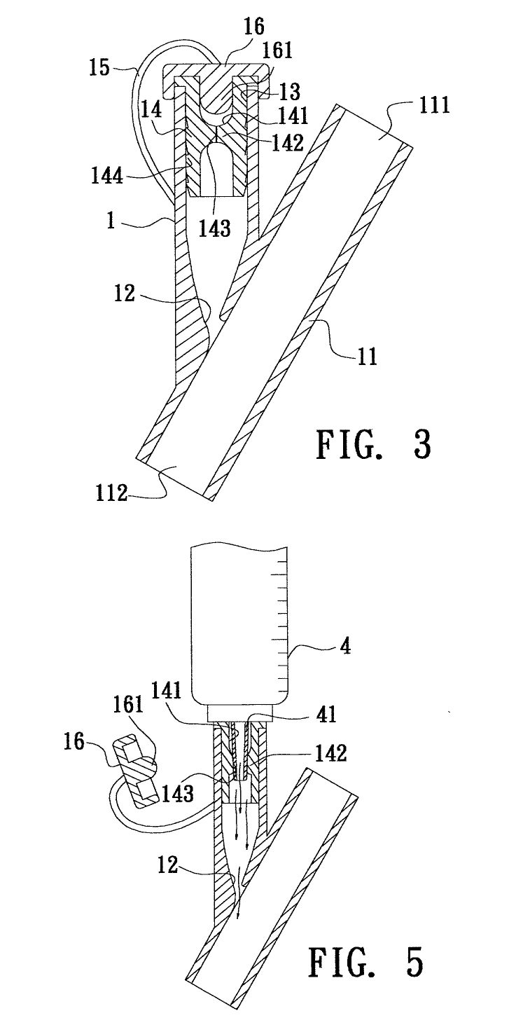

Please refer to FIGS. 1 to 4. The drug-adding tube 1 of infusion set of the present invention has a substantially tubular main body 11. The drug-adding tube 1 obliquely intersects the main body 1. Two ends 111, 112 of the main body 11 are respectively connected with the infusion set 2. The drug-adding tube 1 is formed with a conic neck section 12 which has a tapered inner diameter and is adjacent to the main body 11. The inner diameter of the neck section 12 is approximate to the diameter of a common needle. One end of the drug-adding tube 1 distal from the main body 11 has an opening 13. A rubber-made valve plug 14 is plugged in the opening 13. The valve plug 14 is formed with an insertion port 141 extending from outer side to inner side. A valve body 142 is formed in the valve plug 14 to interrupt the insertion port 141. The center of the valve body 142 is formed with a split 143 passing through the valve body 142. In normal state, due to the valve body's own resilient tension, the split 143 is compressed and sealed. The outer circumference of the valve plug 14 is formed with multiple conic annular ribs 144 for enhancing the tightness of the valve plug 14 plugged in the opening 13 of the drug-adding tube 1. A connecting string 15 integrally connects a cap 16 with the drug-adding tube 1. The cap 16 has a boss section 161 corresponding to the insertion port 141 of the valve plug 14. The boss section 161 can be inserted into the insertion port 141 to block the same.

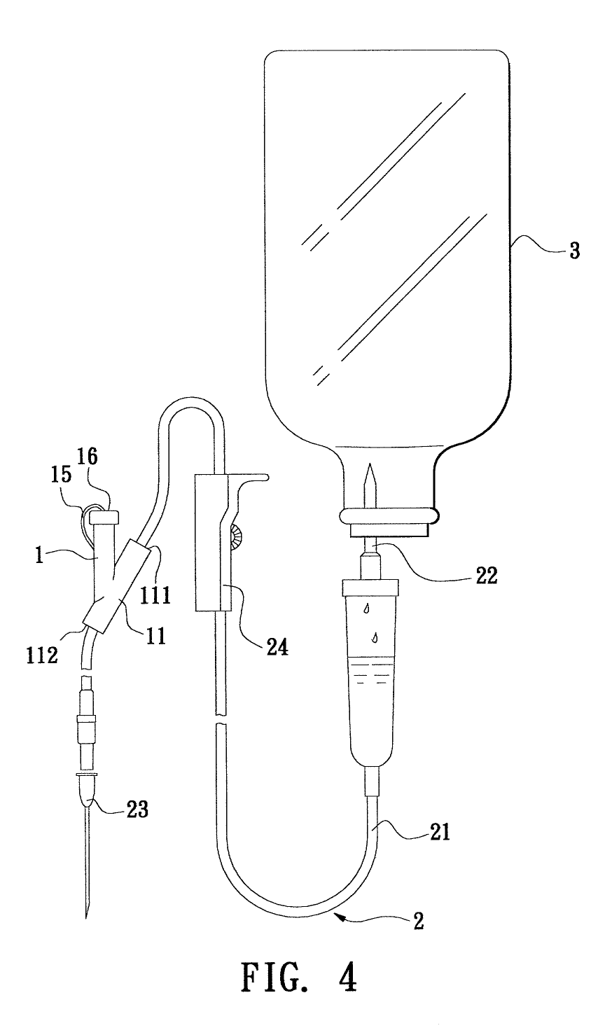

Referring to FIG. 4, the drug-adding tube 1 is disposed in the infusion set 2. The infusion set 2 includes an cannular 21. Two ends of the infusion set 2 are respectively provided with a plastic needle 22 for thrusting into a plastic infusion bag 3 and an intravenous catheter 23 for thrusting into human body. In this embodiment, the drug-adding tube 1 is disposed on a section of the infusion set 11 near the intravenous catheter 23. Two ends 111, 112 of the main body 11 are respectively connected with the infusion set 21. A flow regulator 24 is disposed on a middle section of the infusion set 11.

Referring to FIG. 5, when adding a medicine into the drug-adding tube 1 with a syringe 4, the cap 16 is first taken off and then the front end 41 of the syringe 4 is inserted into the insertion port 141 of the valve plug 14. At this time, the front end 41 of the syringe 4 frees from any needle stretches open the split 143 of the rubber-made valve body 142 to free the valve body 142. Accordingly, the medicine in the syringe 4 can be conveniently injected into the drug-adding tube 1. The neck section 12 of the drug-adding tube 1 has reduced diameter so that a medical worker can operate the syringe 4 in accordance with the original injection mode without the problem of too fast injection. After the medicine is completely injected, the syringe 4 is directly extracted and the originally stretched split 143 will restore to a sealed state due to the valve body's own resilient tension. Therefore, the problem of leakage of medicine is avoided. Then the cap 16 is wiped by an alcohol cotton and sterilized. Then the boss section 161 of the cap 16 is plugged into the insertion port 141 of the valve plug 14 to truly seal the valve plug 14 without contamination. Furthermore, the medicine is added into the drug-adding tube 1 without using any needle so that the problem of impalement of medical worker is avoided and the safety of the medical worker can be ensured. Therefore, the medical worker will not be worried about the problem of impalement and the syringe 4 can be more quickly replaced with another syringe containing different medicine.

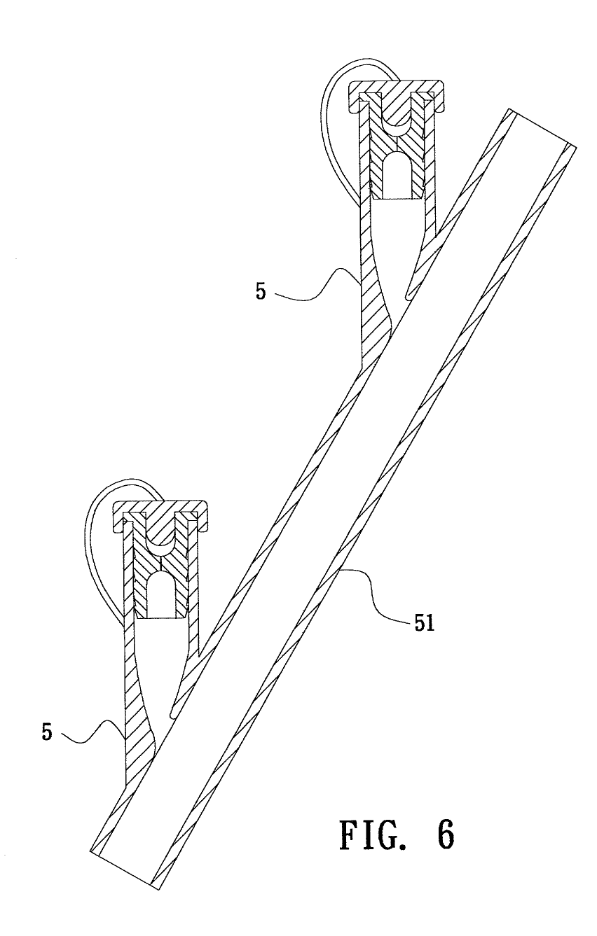

FIG. 6 shows a second embodiment of the present invention, in which two identical drug-adding tubes 1 are disposed on the main body 51. Accordingly, various medicines can be injected at the same time.

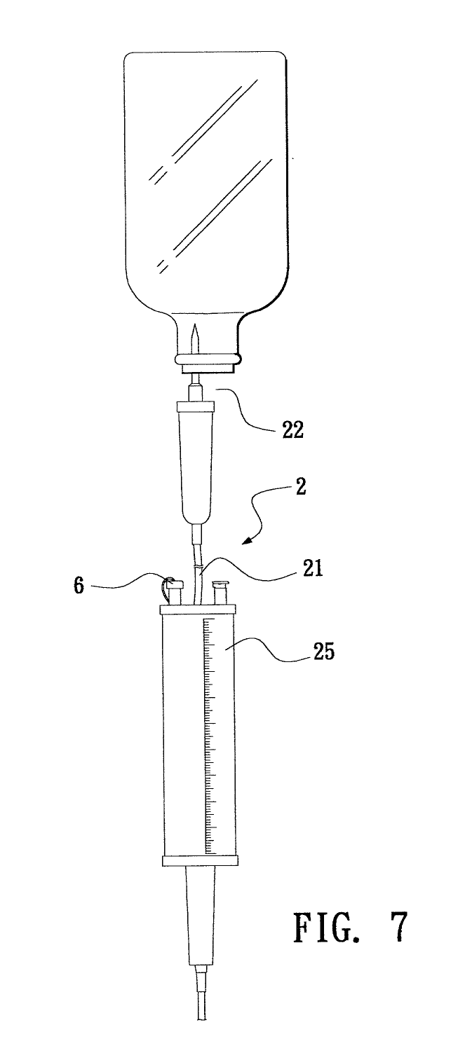

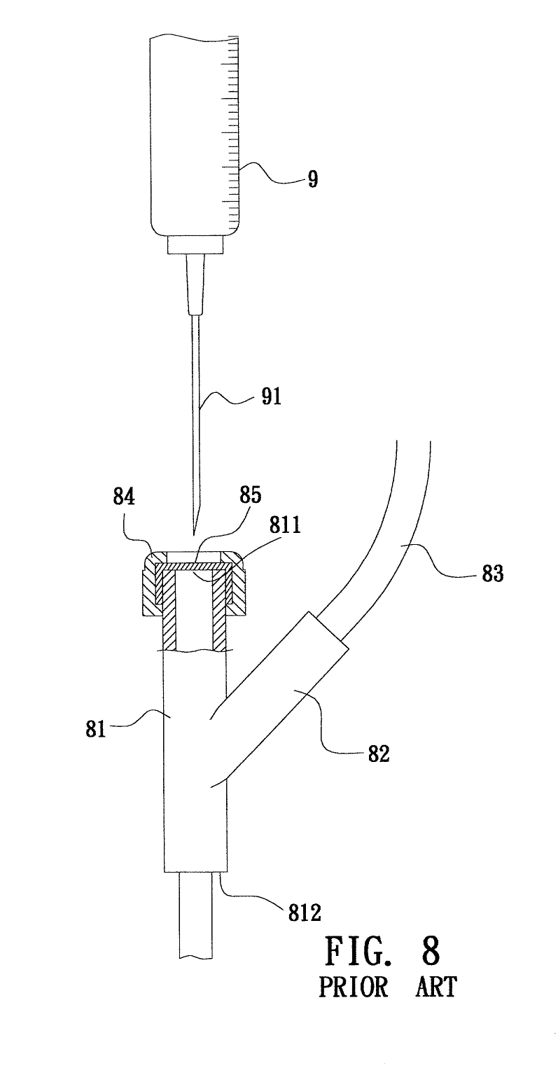

FIG. 7 shows a third embodiment of the present invention, in which the drug-adding tube 6 is directly disposed on a reserving bag 25 near the plastic needle 22 of the infusion set 2. This can achieve the same function as the first embodiment.

The above embodiments are only used to illustrate the present invention, not intended to limit the scope thereof. Many modifications of the above embodiments can be made without departing from the spirit of the present invention.

What is claimed is:

1. A drug-adding tube of infusion set, comprising a substantially tubular main body, two ends of which are respectively connected with the infusion set, at least one drug-adding tube being disposed on the main body and inclined from an axis of the main body, the drug-adding tube being formed with a neck section having reduced inner diameter and being adjacent to the main body, one end of the drug-adding tube distal from the main body having an opening, a rubber-made valve plug being plugged in the opening, the valve plug being formed with an insertion port, a valve body being formed in the valve plug to interrupt the insertion port, a center of the valve body being formed with a split passing through the valve body, whereby in normal state, due to the valve body's own resilience, the split is compressed and sealed, a connecting string integrally connecting a cap with the drug-adding tube, the cap having a boss section corresponding to the insertion port of the valve plug, whereby the boss section can be inserted into the insertion port to block the same.

2. The drug-adding tube of infusion set as claimed in claim 1, wherein the neck section of the drug-adding tube is tapered and has a conic form.

3. The drug-adding tube of infusion set as claimed in claim 1, wherein outer circumference of the valve plug is formed with multiple conic annular ribs for enhancing the tightness of the valve plug plugged in the opening of the drug-adding tube.

4. The drug-adding tube of infusion set as claimed in claim 1, wherein the infusion set has a reserving bag and the drug-adding tube is disposed on the reserving bag.
